(19) United States
(12) Patent Application Publication
(10) Pub. No.: US 2016/0287855 A1
(43) Pub. Date: Oct. 6, 2016

(54) SURGICAL METHODS/DEVICES FOR TISSUE INJURY REMOVAL BY TATTOOING OF AUTOLOGOUS STEM CELLS

(71) Applicants: Alvaro Pacifici, Perugia (IT); Leonardo Cecchetti, Saonara (IT); Walter Cecchetti, Saonara (IT)

(72) Inventors: Alvaro Pacifici, Perugia (IT); Leonardo Cecchetti, Saonara (IT); Walter Cecchetti, Saonara (IT)

(21) Appl. No.: 14/839,950

(22) Filed: Aug. 29, 2015

Related U.S. Application Data
(60) Provisional application No. 62/143,245, filed on Apr. 6, 2015.

(51) Int. Cl. A61M 37/00; A61L 27/36; A61L 27/50

(52) U.S. Cl. CPC A61M 37/0015; A61L 27/50; A61L 27/3695; A61L 27/3604; A61L 27/3691; A61M 37/0084; A61L 2430/34; A61M 2202/08; A61M 2037/0061; A61M 2202/0007; A61M 2202/0014; A61M 2205/7545

(57) ABSTRACT

A method and a device for autografting of fat microparticles containing stem cells of various types, injected into scars, or stretch marks, and/or other cutaneous injuries, at epidermis-dermis level is presented. Fat is taken from the patient in the periumbilical area, where large quantities of mesenchymal stem cells exist. After reduction in size by means of a thin grid emulsifier filter the fat microparticles are loaded into a tank of a multi-needle gun, such as used for tattoos. The multi-needle tattoo gun injects the fractionated fat microparticles into the epidermis-dermis of scars, and/or stretch marks and/or other skin defects or even internal injuries. The injected fat particles trigger tissue regeneration, deleting scars, stretch marks, wrinkles and/or other defects or skin damages in 3-4 weeks. This method for regeneration of tissues can be extended to all surgeries, including vital organs of the human body; always by means of tattooing the target organ with autologous fat microparticles containing stem cells, wherein access is through open, endoscopic, and/or laparoscopic surgery.

### RELATED CASE INFORMATION

This application claims the benefit of U.S. Provisional Application Ser. No. 62/143,245, filed Apr. 6, 2015, entitled “Surgical Method/Device for Tissue Injury Removal, by Autografting Stem Cells Using Tattoo of Autologous Fat Particles” which are incorporated by reference in its entirety herein.

### FIELD OF THE INVENTION

This invention is related to minimally invasive surgical methods involving plastic and/or aesthetic surgery for the deletion of scars, stretch marks, wrinkles and/or other skin injuries and extending to regeneration of all the internal tissues of the human body, including vital organs. More specifically it deals with tattoo-gun systems and a kit-devices, employing only autologous stem cells, extracted from the fat of the patient, collected in their periumbilical area.

### BACKGROUND OF THE INVENTION

The present invention is primarily related to the field of application of Plastic and Aesthetic Surgery. More particularly, the invention relates to scar and other skin defects deletion through stem cells autografting. Moreover the autografting can be extended to the regeneration of tissues in the inside of the body through open surgery or by endoscopic-endocavitary and/or laparoscopic-robotic surgery access techniques.

### Invention Disclosure Statement

Scar deletion—The scar deletion is a complex process that presents various difficulty degrees, depending on scar typology and origin. In order to delete a scar, different kinds of treatments can be employed, but they do not ensure an optimal solution of the problem. Cutaneous defects of scars and stretch marks are the consequences of more or less traumatic events and they can present very different characteristics, with different degrees of visibility.

The attempt to delete scars can be more or less problematic and the methods include medical and surgical procedures.

The most used medical treatments to attempt scar and/or stretch marks deletion, are: a}—occlusion with silica gel sheets or silica gel cream; b}—cortisone injection;

c}—dermabrasion; and d}—laser treatments.

The most used surgical treatments for attempting scars and/or stretch-marks deletion are: a}—intralesional excision; and b}—complete excision.

The kind of technique adopted by the surgeon to delete the scars varies according to the type of scar, to its breadth and to the affected body area. Moreover surgical methods that use autologous tissue (own cells) exist, with partial autografts of human tissues. It must be underlined that the possible surgical options must be accepted by the patient, who has to be informed and to keep in mind that the degree of improvement will be partial. Surgical treatments of scars vary depending on whether they are keloid or hypertrophic scars.

The surgical treatment of the keloid consists in the so called intralesional excision of the formation, which is to say without its complete removal. Such method has a high probability that a keloid of the same dimensions and appearance reforms.

The surgical treatment of the hypertrophic scars consists in the complete excision of the entire scar and also in this case the probability of scar reformation is high, being the subjective characteristics of the patient's skin the determinant element in the onset of pathological scars.

In fact an effective surgical method with good results for scar deletion has not yet been conceived.

Biomedical Tissue engineering—Different methods have been used to obtain tissue regeneration, in vitro and in vivo, the major part of which uses surgery to implant portions of umbilical cords on vital organs. Scientific research on the topic has oriented towards production of new tissues (neo-morphogenesis), a process that leads to, through the specialization of cells, the formation of new tissue and organs both in vitro and in vivo. Rejection is one of the limits to these methods unless autologous (from the same subject) cells are used.

Such techniques are classified as “Biomedical Tissue Engineering”, whose strategies are: 1—use of substances capable of inducing tissue formation; 2—use of isolated cells and in particular stem cells; and 3—Use of cells implanted on matrices or injected into compliant tissues.

Many companies and university research centers have operated in the tissue regeneration field, using stem cells or biomedical tissue engineering. Tissue regeneration of vital organs has been obtained through tissue engineering techniques with substitution of elements with shape memory, as heart valves, and implantation of membranes for bone sealing and regrowth.

These methods are complex, expensive and the cultivation of cells in vitro takes several days to complete and the procedure is not always reliable.

Moreover the grafting of the engineered tissues are generally invasive procedures and require total anesthesia, thus making it impossible to perform outpatient.

Stem cells—The ability of the human body to regenerate and partially renew itself is linked to the possibility to use versatile cells capable of indefinitely multiply, for regenerating all kinds of tissues and repairing lesions in organs in order to bring them back to a normal functioning. Such cells are called stem cells. Autologous stem cells can be administered through autograft techniques. In literature there is a large amount of scientific works and patents on the use and on the possible biomedical applications of stem cells' Stem cells are potentially capable of evolving into any of the over 200 biological structures present in the human body. In order to transform and specialize into a given direction they must be “instructed ” that is to say being subjected to definite conditions or stimuli: in this area many and important biomedical research activities have started see for example—US 2009/0317367 A1 by Chazenbalk et al. (43): “Method of producing preadipocytes and increasing the proliferation of adult adipose stem/progenitor cells”. Dec. 24, 2009; U.S. Pat. No. 7,153,684 (2006) by Hogan B L M,” Pluripotential embryonic stem cells and methods of making same”; U.S. Pat. No. 7,256,042 (2007) by Rambhatla L, et al., “Process for making hepatocytes from pluripotent stem cells”; U.S. Pat. No. 7,727,762 (2010) by Fukuda K, Yuasa S, Okano H, Shimazaki T, Koshimizu U, Tanaka T, Sugimura K., “Method of inducing the differentiation of stem cells into myocardial cells” {hereinafter, Fukuda); and U.S. Pat. No. 7,763,466 (2010) by Keller G M, Kouskoff V, et al. “Mesoderm and definitive endoderm cell populations”.

According to their capability to evolve, stem cells are classified as:
totipotent: unlimited differentiation capability from the embryo to the adult body;
pluripotent: differentiation capability into cells belonging to any of the three germ layers (adipocytes);
multipotent: differentiation capability into multiple, but limited, cell types (hematopoietic cells);
unipotent: differentiation capability into a single kind of cells (osteoblasts).

Recent technical and scientific progress in biomedical tissue engineering has reached a state of the art capable of producing matrices for supporting the proliferation and differentiation of stem cells, able to produce or regenerate many different tissues of the human body. This paves the way to noteworthy developments of biomedical science. Depending on their origin, stem cells are distinguished between adult, fetal, umbilical cord, and embryonic cells. Adult stem cells (ASCs) can be extracted, isolated and also cultivated in the laboratory; recent biological research has demonstrated that they can also originate different tissues; in this case they can be classified as pluripotent. Recently very positive experimental results have been obtained with mesenchymal ASCs on patients affected by cardiac ischemia (Fukuda).

The current state of the art focuses on the extraction and isolation of stem cells from areas like the umbilical cord or the bone marrow, which present many shortcomings, i.e. the limited or no-availability for the umbilical cord cells, and the invasiveness of the extraction process for the bone marrow cells (therefore no outpatient treatment possible). Another option if to extract stem cells from embryos, a procedure that presents many ethical and legal issues in many countries.

The methods that employ heterologous stem cells can encounter problems related to compatibility of tissues between different patients.

Another approach consists in the reprogramming of non-stem cells into stem cells, through complex bio-chemical treatments of cells used. These methods bypass the issue of extracting stem cells, but have the disadvantage of the high costs and the complexity of the process, at this time only few laboratories have the capability to perform them. Commercial success and utility are not discernable yet.

Due to the disadvantages and the deficiencies of current tissue regeneration and scar removal techniques, be they through surgery, tissue engineering or extraction/cultivation and use of stem cells, a need exists for a method and device that provides a low invasive, effective and more economical alternative to address their shortcomings.

### Objectives and Brief Summary of the Invention

It is an objective of the present invention to provide a more effective method and device for the regeneration of scars, stretch-marks, wrinkles and/or other skin lesions through a tattoo of microparticles of fat extracted in the periumbilical area of the patient.

It is another objective of the present invention to provide a method for the regeneration of skin injuries through an autograft of autologous stem cells extracted from the fat extracted from the fat withdrawn in the periumbilical area of the patient.

It is yet another objective of the present invention to provide a more effective method and device for the regeneration of tissues inside the human body trough a tattoo of autologous stem cells extracted from the fat withdrawn in the periumbilical area of the patient.

It is still another objective of the present invention to provide a kit-device that allows to extract the fat from the patient, to fraction the fat taken and make it injectable by micro-needles.

It is a further objective of the present invention to provide a tattoo device to inject the microparticles of fat into the scars and/or stretch-marks and/or wrinkles and/or other skin lesions.

It is another objective of the present invention to provide a multi-needle injection device that works similarly to a tattoo gun but is conceived for laparoscopic-robotic applications.

It is another objective of the present invention to provide a multi-needle injection device that works similarly to a tattoo gun but is conceived for endoscopic applications (FIG. 6, 7).

Briefly stated, the present invention provides a device and a method for the regeneration of skin and/or tissues inside the body to repair them. More specifically, a device and a method for autografting of fat microparticles containing stem cells of various types, by injecting into scars, or stretch marks, and/or other cutaneous injuries, at epidermis-dermis level is presented. Fat is taken from the patient in the periumbilical area, where large quantities of mesenchymal stem cells exist. After reduction in size by means of a thin grid emulsifier filter the fat microparticles are loaded into a tank of a multi-needle gun, such as used for tattoos. The multi-needle tattoo gun injects the fractionated fat microparticles into the epidermis-dermis of scars, and/or stretch marks and/or other skin defects or even internal injuries. The injected fat particles trigger tissue regeneration, deleting scars, stretch marks, wrinkles and/or other defects or skin damages in 3-4 weeks. This device and method for regeneration of tissues can be extended to all surgeries, including vital organs of the human body; by means of tattooing the target organ with autologous fat microparticles containing stem cells, wherein access is through open, endoscopic, and/or laparoscopic surgery.

### BRIEF DESCRIPTION OF FIGURES AND PHOTOS

FIG. 1 illustrates injection of pigments and/or fat microparticles into the tissues.

FIG. 2 describes a repair mechanism of the stem cells injected into scars and/or stretch-marks.

FIG. 3 presents a radio frequency head, size 1 cm² matrix, with 7×7=49 micro-needles for radiofrequency, used for stimulation of tissues in aesthetic treatments.

FIG. 4 illustrates a gold plated cannula for scraping fat, two luer-lock syringes and a thin grid filter.

FIG. 5 shows motorized tattoo gun with multi-needles tips type 5 and 9 magnum.

FIG. 6 illustrates a preferred embodiment of the present invention for laparoscopic-robotic treatments.

FIG. 7 presents a preferred embodiment for endoscopic-endocavitary treatments.

FIG. 8 shows the gold plated scraping cannula connected to a luer-lock syringe during fat suction and withdrawal around the umbilicus of a female patient.

FIG. 9 fragmentation of the fat with the thin grid filter.

FIG. 10 separation of the aqueous serum from the composition through emulsion.

In FIG. 11 are before and after photos of patient after use of embodiments of the present invention in mastoplasty surgery.

FIG. 12 is a photo of the patient suffering from evident stretch-marks before the procedure and 4 weeks after the treatment.

FIG. 13 is a photo of the patient suffering from a very deep scar on the face before the procedure, and after 2 treatments executed at a distance of one month each, on the deep lesion on the face.

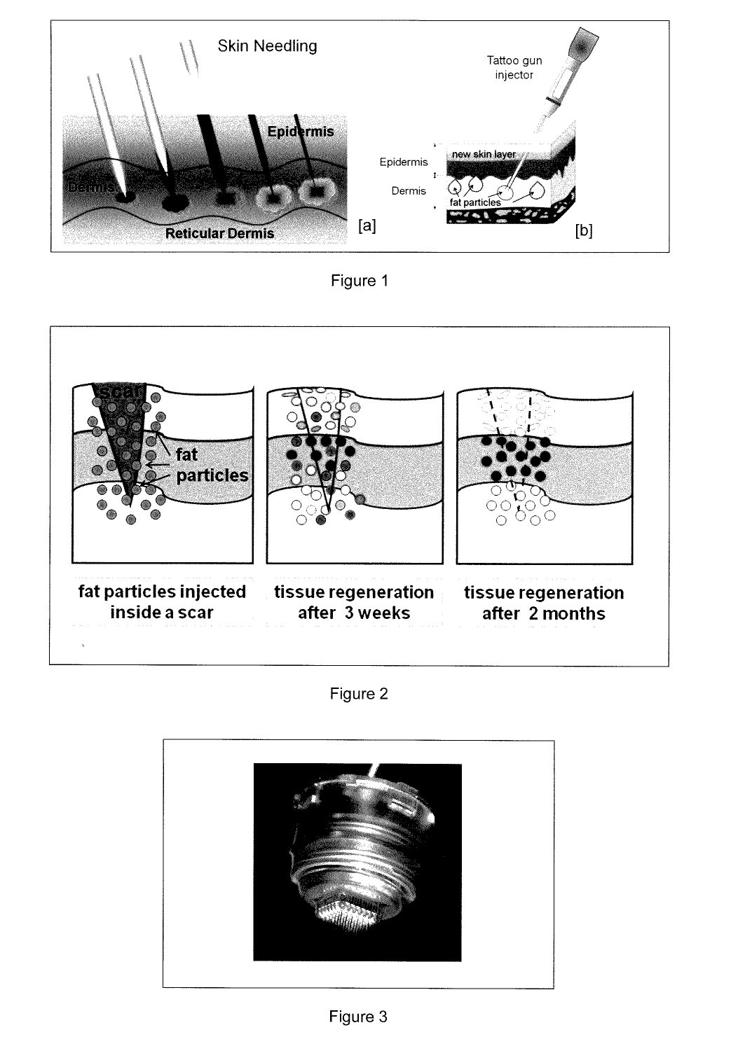

### DETAILED DESCRIPTION OF PREFERRED EMBODIMENTS

The limitations and shortcomings of the prior art techniques can be overcome, as described in this invention, by a stem cells autografting technique, which utilizes autologous fat microparticles containing stem cells, extracted from the surroundings of the umbilicus of the patient, and injected into the skin injuries at epidermis and dermis level, with a tattoo-like device and technique.

This technique, as explained further below, presents some important advantages compared to previous methods, i.e. there are no scar reforming, no rejection issues, an easy and short learning curve, lower invasiveness, possibility to be performed outpatient without anesthesia and at lower costs.

Of particular and surprising note, this technique is not limited to the regeneration of skin injuries, but it can be extended to the regeneration of tissues inside the human body through open surgery or by endoscopic-endocavitary surgery and/or laparoscopic-robotic access techniques.

The intervention consists in the grafting of autologous stem cells into the treatment site.

The deletion of the scars or the repair of the diseased tissues is thus carried out through tissue regeneration by the autologous stem cells implanted.

The present invention discloses an improved method and device for a safe and efficient tissue regeneration though autografting of fat microparticles (containing stem cells), extracted in the surroundings of the umbilicus of the patient and injected into the tissue injuries, as for a tattoo.

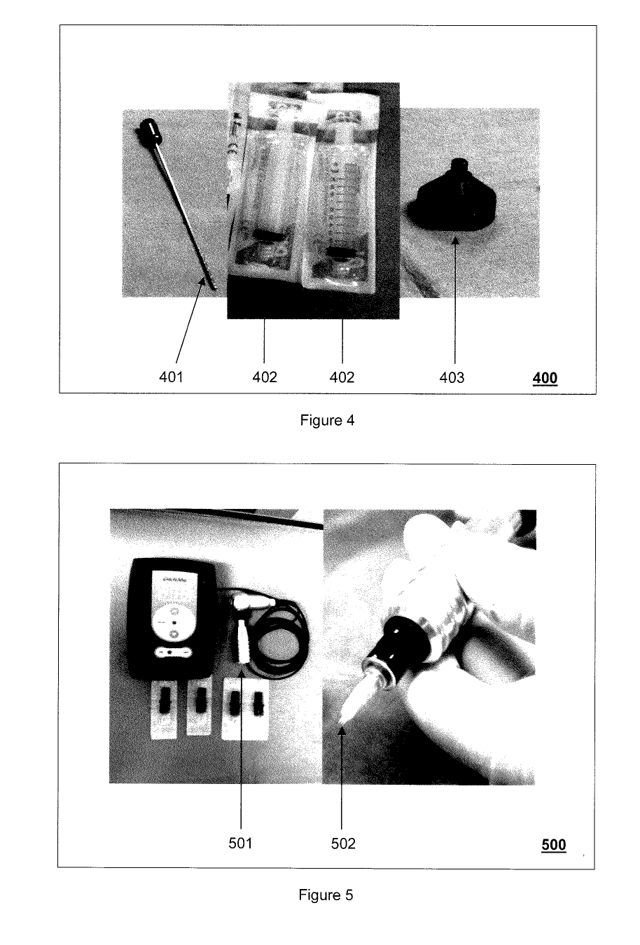

The kit-device for extracting and processing the fat from the periumbilical area of the patient comprises: a specific gold plated cannula for fat withdrawal (sizes: 2.4×150 mm, 4=0.8 mm), two luer-lock syringes and a thin grid emulsifier filter (FIG. 4).

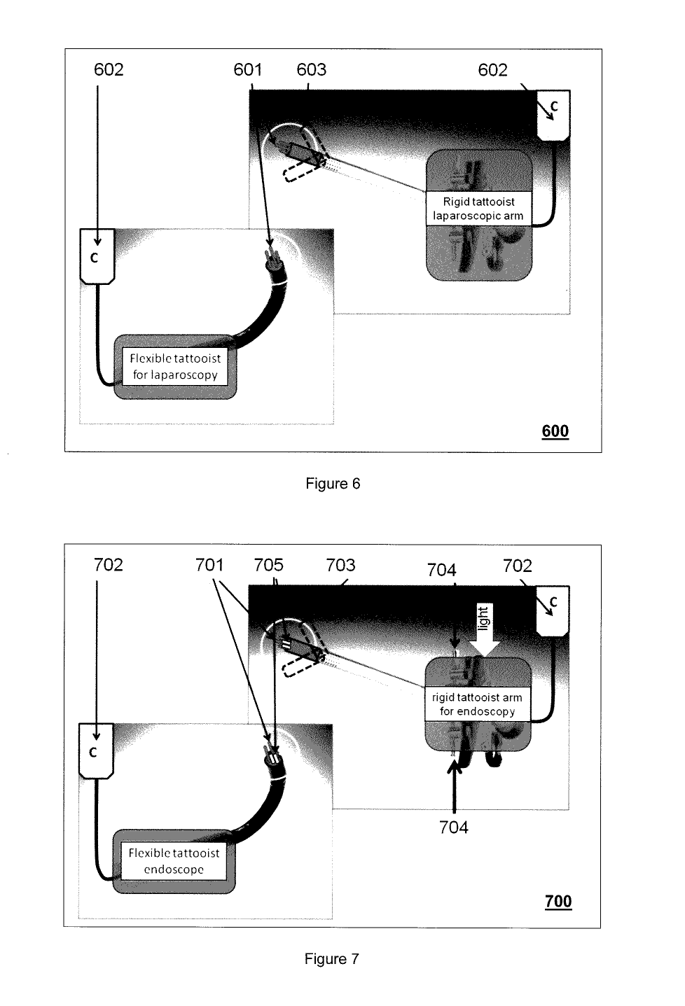

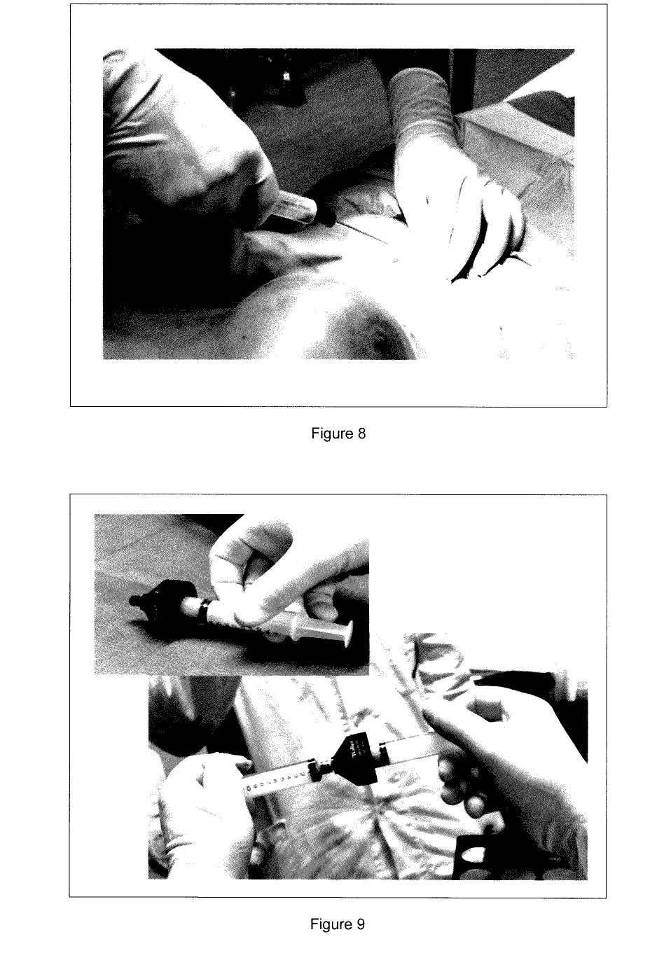

The device for the injection of the composition 1000 of micro-particles of fat extracted from the periumbilical area of the patient, containing autologous stem cells, comprises a multi-needled, channeled needle device to introduce into the chosen treatment site the composition 1000. This device is specific, according to the treatment to be performed: a high quality tattoo gun with one multi-needle injector tip for tattooing, model 5 and 9 magnum for skin injuries treatment; a device with flexible or rigid arm for laparoscopic-robotic surgery and open surgery (FIG. 6); a device with flexible or rigid arm for endoscopic-endocavitary surgery (FIG. 7). All the instrumentation and the devices are sterilized pursuant to a surgical use.

The ability of the human body to regenerate and partially renew itself is linked to the possibility to use versatile cells capable of indefinitely multiply, for regenerating all kinds of tissues and repairing lesions in organs in order to bring them back to a normal functioning. Such cells are called stem cells.

According to their capability to evolve, stem cells are classified as:
totipotent: unlimited differentiation capability from the embryo to the adult body;
pluripotent: differentiation capability into cells belonging to any of the three germ layers (adipocytes);
multipotent: differentiation capability into multiple, but limited, cell types (hematopoietic cells);
unipotent: differentiation capability into a single kind of cells (osteoblasts).

Stem cells are fundamental in the first stages of embryonic development, however multipotent cells are present in the organism of both children and adults, for example bone marrow stem cells that produce red blood cells, white blood cells, platelets, osteoblasts, fibroblasts and adipocytes (in other words, fat cells). Furthermore there are satellite stem cells positioned in the terminations of skeletal muscle fibers capable of repairing injuries by differentiating into myoblasts that will then form multinucleate skeletal muscle cells.

Depending on their origin, stem cells are distinguished between adult, fetal, umbilical cord, and embryonic cells. Recent biological research has demonstrated that adult stem cells (ASCs) can transform into many types of cells, thus being classified as pluripotent. An important, but relatively unused currently, source of ASCs available in the human body is adipose tissue, particularly positioned in periumbilical area. Fat is thus an accessible source of autologous mesenchymal adult stem cells in the human body. As a consequence, we have found that, in order to obtain tissue regeneration, a feasible way is an autograft of microparticles of fat taken from the periumbilical area, containing adult pluripotent stem cells.

We found withdrawal of adipose tissue can be performed simply and painlessly, also outpatient; and withdrawn fat can be treated, in order to make it injectable together with the stem cells present.

We surprisingly found that the injection method by tattooing is the best grafting system, i.e. most efficient and simple, for injecting stem cells inside the tissues to be regenerated, because unexpectedly it allows to inject stem cells at the depth and in the ideal quantity for optimally regenerating tissues.

### Example 1. Treatment of Skin Lesions

Schematically, FIG. 4 and FIG. 5 depict a preferred embodiment in which tissue regeneration is performed by two kit-devices (400 and 500), one for fat extraction and filtering, and the other for grafting the fat microparticles obtained. They comprise gold plated cannula for fat withdrawal 401, two luer-lock syringes 402, thin grid emulsifier filter 403, tattoo gun system 501, multi-needle injector tip 502 of the tattoo gun, model 5 magnum and 9 magnum.

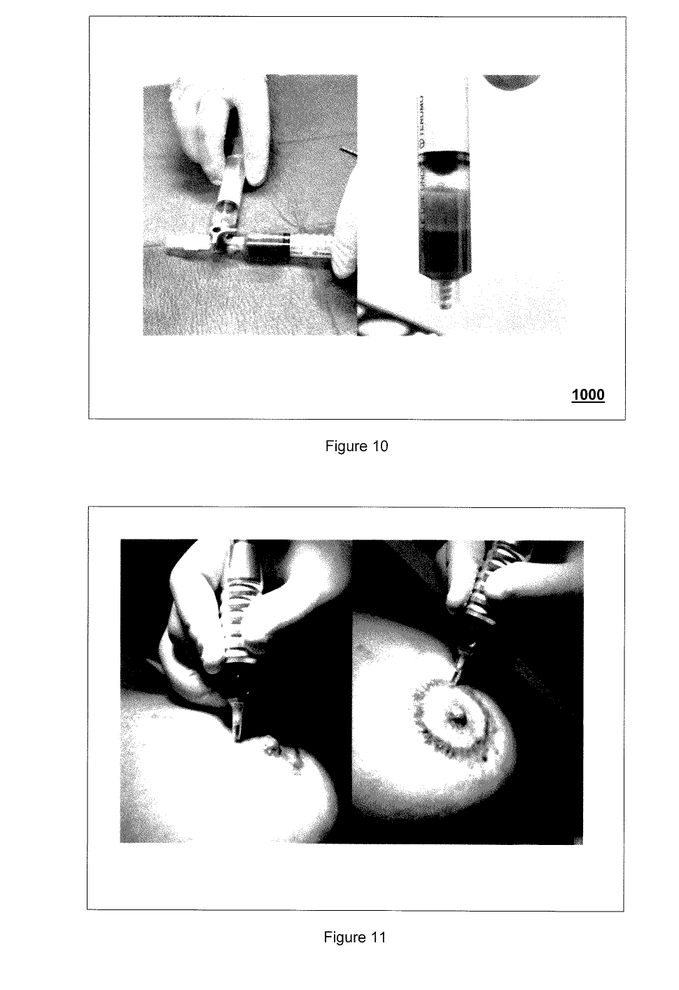

Around the umbilicus of the patient, under topical sedation, a withdrawal of the fat is performed with cannula 401, connected to a syringe 402, which suctions the fat (FIG. 8). This cannula is provided with gaping holes that scrape the fat where pluripotent and unipotent stem cells are present. Subsequently the syringe 402, filled with fat, is connected to filter 403. By connecting another syringe 402 on the other side of the filter 403, the fat is pumped through the filter 403 several times, passing from one syringe 402 to the other, thus being fragmented by the thin grid until it is decomposed into an injectable micro-particles composition (FIGS. 10-1000). After the fat fragmentation, the fat micro-particles composition 1000 is separated from the serum through decantation (FIG. 10); such composition 1000 contains a high number of stem cells.

The composition 1000 is placed into the tank of the tattoo gun 501, provided with tip 502. By using the multiple syringes tip 502 of the gun 501, the fat particles are then injected into the scars and/or stretch marks and/or other cutaneous lesions by using the same procedures of tattooing, where instead of pigments fat particles containing stem cells are injected.

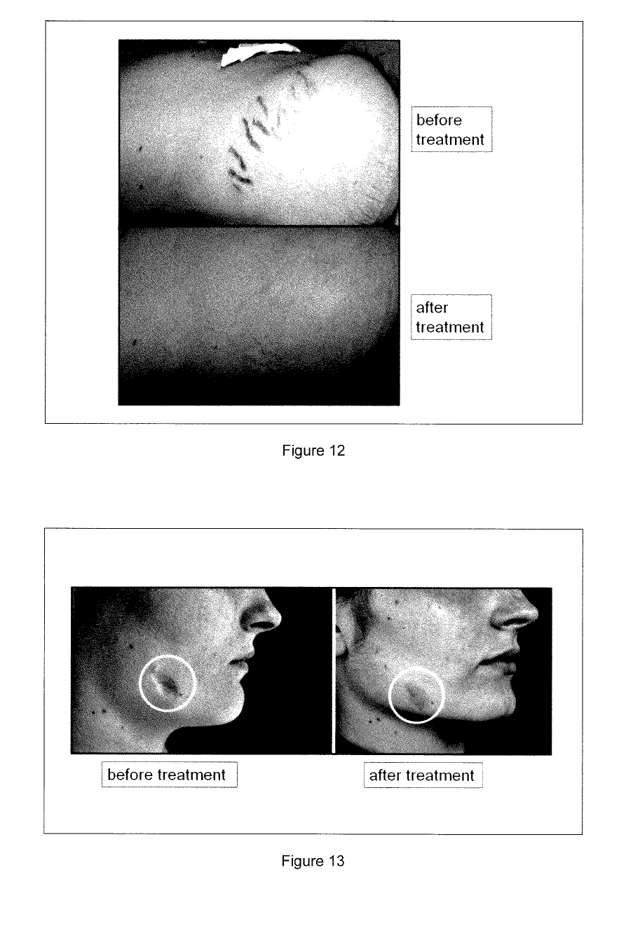

After 4 weeks the effects caused by tissue regeneration that delete scars are already evident (FIG. 12, FIG. 13); however, in order to obtain the best results, the procedure must be repeated twice at a distance of 3 to 4 weeks. In more compromised cases (FIG. 13), also a third treatment can be performed without contraindications.

### Example 2 Treatment of Tissues Inside the Body Through Laparoscopy/Robotics

FIG. 4 and FIG. 6 depict a preferred embodiment in which tissue regeneration is performed by two kit-devices (400 and 600), one for fat extraction and filtering, and the other for grafting the fat microparticles obtained. They comprise gold plated cannula for fat withdrawal 401, sterile hollow container to collect fat taken from the patient 402, two luer-lock syringes 402, thin grid emulsifier filter 403, multi-needle injection device 600 that works similarly to a tattoo gun but is conceived for laparoscopic applications, provided with multi-needle motorized tips 601, small cruet to be filled with stem cells 602 and orientable tip on the tattooist laparoscopic arm 603.

Around the umbilicus of the patient, under topical sedation, a withdrawal of the fat is performed with cannula 401, connected to a syringe 402, which suctions the fat (FIG. 8). This cannula is provided with gaping holes that scrape the fat where pluripotent and unipotent stem cells are present. Subsequently the syringe 402, filled with fat, is connected to filter 403. By connecting another syringe 402 on the other side of the filter 403, the fat is pumped through the filter 403 several times, passing from one syringe 402 to the other, thus being fragmented by the thin grid until it is decomposed into an injectable micro-particles composition (FIGS. 10-1000). After the fat fragmentation, the fat micro-particles composition 1000 is separated from the serum through decantation (FIG. 10); such composition 1000 contains a high number of stem cells.

The composition 1000 is placed into the cruet 602 of the injection device 600. By using the multiple syringes tip 601 of the device 600, the fat particles are then injected into the diseased, damaged, worn or aged tissue to be treated through laparoscopy inside the body by using a procedure similar to that of tattooing, where instead of pigments fat particles containing stem cells are injected.

### Example 3 Treatment of Tissues Inside the Body Through Endoscopy and/or Endocavitary

FIG. 4 and FIG. 7 depict a preferred embodiment in which tissue regeneration is performed by two kit-devices (400 and 700), one for fat extraction and filtering, and the other for grafting the fat microparticles obtained. They comprise gold plated cannula for fat withdrawal 401, two sterile luer-lock syringes 402, thin grid emulsifier filter 403, multi-needle injection device 700 that works similarly to a tattoo gun but is conceived for endoscopic applications, provided with multi-needle motorized tips 701, small cruet to be filled with stem cells 702, orientable tip on the tattooist endoscopic arm 703, irrigation channel 704 and integrated lighting and camera systems 705.

Around the umbilicus of the patient, under topical sedation, a withdrawal of the fat is performed with cannula 401, connected to a syringe 402, which suctions the fat (FIG. 8). This cannula is provided with gaping holes that scrape the fat where pluripotent and unipotent stem cells are present. Subsequently the syringe 402, filled with fat, is connected to filter 403. By connecting another syringe 402 on the other side of the filter 403, the fat is pumped through the filter 403 several times, passing from one syringe 402 to the other, thus being fragmented by the thin grid until it is decomposed into an injectable micro-particles composition (FIGS. 10-1000). After the fat fragmentation, the fat micro-particles composition 1000 is separated from the serum through decantation (FIG. 10); such composition 1000 contains a high number of stem cells.

The composition 1000 is placed into the cruet 702 of the injection device 700. By using the multiple syringes tip 701, the fat particles are then injected into the diseased, damaged, worn or aged tissue to be treated through endoscopy inside the body by using a procedure similar to that of tattooing, where instead of pigments fat particles containing stem cells are injected.

### Example 4 Treatment of Tissues Inside the Body Through Open Surgery

FIG. 4 and FIG. 6 depict a preferred embodiment in which tissue regeneration is performed by two kit-devices (400 and 600), one for fat extraction and filtering, and the other for grafting the fat microparticles obtained. They comprise gold plated cannula for fat withdrawal 401, sterile hollow container to collect fat taken from the patient 402, two luer-lock syringes 402, thin grid emulsifier filter 403, multi-needle injection device 600 that works similarly to a tattoo gun but is conceived for laparoscopic applications, provided with multi-needle motorized tips 601, small cruet to be filled with stem cells 602 and orientable tip on the tattooist laparoscopic arm 603. The laparoscopic device is preferred in open surgery applications over the tattoo-gun because of its longer arm that allows to more easily reach areas inside the human body.

Around the umbilicus of the patient, under topical sedation, a withdrawal of the fat is performed with cannula 401, connected to a syringe 402, which suctions the fat (FIG. 8). This cannula is provided with gaping holes that scrape the fat where pluripotent and unipotent stem cells are present. Subsequently the syringe 402, filled with fat, is connected to filter 403. By connecting another syringe 402 on the other side of the filter 403, the fat is pumped through the filter 403 several times, passing from one syringe 402 to the other, thus being fragmented by the thin grid until it is decomposed into an injectable micro-particles composition (FIGS. 10-1000). After the fat fragmentation, the fat micro-particles composition 1000 is separated from the serum through decantation (FIG. 10); such composition 1000 contains a high number of stem cells.

The composition 1000 is injected into the cruet 602 of the injection device 600. By using the multiple syringes tip 602 of the device 600, the fat particles are then injected into the diseased, damaged, worn or aged tissue to be treated through open surgery inside the body by using a procedure similar to that of tattooing, where instead of pigments fat particles containing stem cells are injected.

Having described preferred embodiments of the invention with reference to the accompanying drawings, it is to be understood that the invention is not limited to these precise embodiments, and that various changes and modifications may be effected therein by one skilled in the art without departing from the scope or spirit of the invention as defined in the appended claims, including changes in application or method of use or operation, method of manufacture, shape, size, or material which are not specified within the detailed written description or illustrations contained herein yet would be apparent or obvious to one skilled in the art.

What is claimed is:

1. A tissue repair and rejuvenation method, at a preselected treatment site, using a composition of microparticles of adipose tissue, containing autologous stem cells, comprising the steps of:
a) extracting some of a patient's adipose tissue in the periumbilical area,
b) micro-fragmenting of the adipose tissue, to make it injectable,
c) filtering and isolating of stem cells, from the adipose tissue,
d) loading a composition of microparticles of the filtered adipose tissue into channels of a multi-needled, channeled device,
e) injecting said adipose microparticle composition, using a tattoo-like procedure, into said preselected treatment site, and
f) repeating steps d) and e) as necessary to fully treat the pre-selected tissue treatment site/area.

2. The regenerative-tattoo method according to claim 1, wherein said step c), filtering and isolating of stem cells, comprises fragmenting the adipose composition through at least one thin grid filter.

3. The regenerative-tattoo method according to claim 1, wherein said filtering and isolating step further reduces in size the adipose tissue particles to facilitate steps d) and e).

4. The regenerative-tattoo method according to claim 1, wherein the tissue to be repaired/rejuvenated is a scar, a stretch mark, wrinkles or another type of skin lesion.

5- The regenerative-tattoo method according to claim 1, wherein the tissue to be repaired/rejuvenated belongs to any part of the human body, including any internal organ.

6. A device system for tissue repair and rejuvenation, using the regenerative-tattoo method according to claim 1, comprising:
a) means for adipose tissue (fat) extraction,
b) at least one syringe, wherein said syringe is selected from a group of a luer-lock syringe and a standard syringe,
c) at least one thin grid filter to micro-fragment the adipose particles,
d) a second means to select and extract stem cells from the fragmented adipose particles,
e) a container to hold a composition of micro-fragmented adipose particles containing autologous stem cells, and
f) a multi-needled, channeled needle device.

7. The device system for tissue repair and rejuvenation according to claim 6, wherein said channeled needle device is a tattoo gun.

8. The device system according to claim 6, wherein said channeled needle device is selected from the group consisting of a tattoo device provided with single/multiple needles, tips, blades, a sputtering device, and a deposition/coating device, for handling adipose microparticle compositions and isolated stem cells.

9. The device system according to claim 6, wherein said channeled needle device is selected from a group consisting of an endoscopic-endocavitary device system and a laparoscopic-robotic device system, provided with micro-needle tips analogous to tattoo guns.

10. Laparoscopic-robotics device systems according to claim 9, having arms, provided with rigid of tiltable extremities with micro-needles devices on the tip, capable of tattooing microparticle compositions into tissues of the human body with an integrated/external cruet for containing microparticle compositions.

11. The laparoscopic-robotics device systems according to claim 10, wherein said arms are selected from the group of rigid arms, and flexible arms.

12. The laparoscopic-robotics device systems according to claim 11 where said micro-needles devices are at least similar to those used by the tattoo-guns used for traditional tattoos.

13. The laparoscopic-robotics device systems according to claim 11 where said rigid/flexible arms devices are provided with handpieces used for the management of the device, 14- The laparoscopic-robotics device systems according to claim 11 where said micro-needle device has command for its functioning present on a component selected from the group consisting of a handpiece, a footswitch and any other device in the system.

15. The laparoscopic-robotics device systems according to claim 11, wherein said rigid/flexible arms are provided with at least one operating channel.

16. The laparoscopic-robotics device systems according to claim 15 wherein said channel has a use selected from a group consisting of a flow of a fluid, and vision with cameras, positioned in any part of the system.

17. Endoscopic-endocavitary device systems according to claim 9 having rigid/flexible arms with rigid tiltable extremities with micro-needles devices on the tip, capable of tattooing microparticle compositions into the tissues of a human body and with an integrated/external cruet for containing the microparticle composition.

18. The endoscopic-endocavitary device systems according to claim 17 where said micro-needles devices are at least similar to those used by the tattoo-guns used for traditional tattoos.

19. The endoscopic-endocavitary device systems according to claim 17 wherein said rigid/flexible arms devices are provided with handpieces used for the management of the device, using single/multiple parts, which are linked or independent.

20. The endoscopic-endocavitary device systems according to claim 17 where said micro-needle device has command for its functioning present on a component selected from the group consisting of a handpiece, a footswitch and any other device in the system.

21. The endoscopic-endocavitary device systems according to claim 17 where said rigid/flexible arms are provided with at least one operating channel,

22. The endoscopic-endocavitary device systems according to claim 21, wherein said channel has a use selected from a group consisting of a flow of a fluid, and vision with cameras, positioned in any part of the system.
